(19) United States
(12) Patent Application Publication
(10) Pub. No.: US 2023/0263755 A1
(43) Pub. Date: Aug. 24, 2023

(54) STABILIZATION OF ACETAMINOPHEN IN AQUEOUS SOLUTION

(71) Applicant: Nostrum Pharmaceuticals, LLC, Somerset, NJ (US)

(72) Inventor: Nirmal V. MULYE, Miami, FL (US)

(73) Assignee: Nostrum Pharmaceuticals, LLC, Somerset, NJ (US)

(21) Appl. No.: 18/173,349

(22) Filed: Feb. 23, 2023

Related U.S. Application Data
(60) Provisional application No. 63/313,181, filed on Feb. 23, 2022.

(51) Int. Cl. A61K 31/196; A61K 47/12; A61K 31/485

(52) U.S. Cl. CPC A61K 31/196; A61K 47/12; A61K 31/485

(57) ABSTRACT

Stable pharmaceutical solution comprising acetaminophen and method of making such are provided. The pharmaceutical solution includes: an aqueous solution comprising acetaminophen; a solubilizing effective amount of a solubilizer, a weak organic acid having a having a having a COOH group and pKa greater than 3. The weak acid is in a sufficient amount to achieve and maintain the pharmaceutical solution at a pH from about 3.8 up to 6.61 for stability of acetaminophen and maintain effectiveness of the preservative. The method of making a stable pharmaceutical solution comprising acetaminophen includes: mixing water, acetaminophen, a solubilizing effective amount of a solubilizer, and a weak organic acid having a having a COOH group and pKa greater than 3 in a sufficient amount to form and maintain the solution at a pH from about 3.8 up to 6.61 for stability of acetaminophen.

### FIELD OF THE DISCLOSURE

The present disclosure relates to a pharmaceutical solution having acetaminophen in an aqueous solution. More specifically, the invention relates to an aqueous pharmaceutical solution comprising acetaminophen stabilized with a weak organic acid having a pKa greater than or equal to 3.0.

### BACKGROUND

Acetaminophen, also known as paracetamol (chemically known as N-(4-hydroxyphenyl)acetamide or N-acetyl-4-aminophenol) is usually well tolerated by human beings and lacks many of the undesired effects of other analgesics. Therefore, acetaminophen is often used to help relieve pain, fever, inflammation, ischemic injury and a range of other diseases and conditions in adults, children elderly; and treat various diseases

Although several pharmaceutical preparations with acetaminophen in powder, tablet, granule, or topical administration are known, it is still difficult to obtain a stable aqueous pharmaceutical solution with acetaminophen because of several disadvantages.

A disadvantage is acetaminophen is not very soluble in water (about 1.3 g per 100 mL). Furthermore, pharmaceutical solutions with acetaminophen are unstable in the presence of oxygen and/or light and the acetaminophen degrades through several degradation pathways. Degradation is visible because the degradation substances itself causes a coloring in the solution; and furthermore, some of the degradation substances causing the coloring, such as benzoquinoimines, are also hepatotoxic. Another disadvantage is acetaminophen is also susceptible to oxidation in aqueous solutions; and require antioxidants for stability, in which some anti-oxidants itself is toxic. Furthermore, oxidation leads to the formation of colored compounds, making the aqueous solution unsuitable for therapeutic use as presence of color itself indicates degradation (supra). Additionally, acetaminophen has a relatively short half-life of about 2 hours.

Although various compositions with acetaminophen in powder or granule form in suspended form or dissolved in a suspending system with xanthan gum, microcrystalline cellulose, etc. have been developed, acetaminophen is still not dissolved or dispersed at a molecular level in a solution. Consequently, what is still needed are stable pharmaceutical solution comprising acetaminophen in aqueous solution and method of making such.

### SUMMARY

The present invention meets these and other needs by providing a pharmaceutical solution comprising acetaminophen in an aqueous solution that is stable and minimizes degradation of acetaminophen and method of making such.

Accordingly, an aspect of the invention provides a pharmaceutical solution comprising acetaminophen. The pharmaceutical solution includes: acetaminophen in an aqueous solution that is stable and has a pH from about 3.8 up to 6.1; a sufficient amount of a solubilizer; and a weak organic acid having a pKa greater than 3. The weak acid is in a sufficient amount to achieve and maintain the pharmaceutical solution at a pH from about 3.8 up to 6.1 for stability of acetaminophen.

A second aspect of the invention provides a pharmaceutical solution comprising acetaminophen. The pharmaceutical solution includes: an aqueous solution comprising acetaminophen; a solubilizing effective amount of a solubilizer, a weak organic acid having a having a having a COOH group and pKa greater than 3. The weak acid is in a sufficient amount to achieve and maintain the pharmaceutical solution at a pH from about 3.8 up to 6.61 for stability of acetaminophen and maintain effectiveness of the preservative.

A third aspect of the invention provides a method of making a pharmaceutical solution comprising acetaminophen in an aqueous solution that is stable. The method includes: providing water, acetaminophen, a solubilizing effective amount of a solubilizer, and a weak organic acid having a having a COOH group and pKa greater than 3. The weak organic acid is provided in sufficient amount to form and maintain the solution at a pH from about 3.8 up to 6.61 for stability of acetaminophen.

A fourth aspect of the invention provides a method of making a stable pharmaceutical solution comprising acetaminophen. The method includes: mixing water, acetaminophen, a solubilizing effective amount of a solubilizer, and a weak organic acid having a having a COOH group and pKa greater than 3 in a sufficient amount to form and maintain the solution at a pH from about 3.8 up to 6.61 for stability of acetaminophen.

A fifth aspect of the invention provides a pharmaceutical solution comprising acetaminophen. The pharmaceutical solution includes: acetaminophen in an aqueous solution having a pH from about 3.8 up to 6.1; a sufficient amount of a solubilizer; and a weak organic acid having a COOH group and pKa greater than 3. The weak acid is in a range from about 0.01 wt % to about 0.8 wt % of the pharmaceutical solution.

A sixth aspect of the invention provides a pharmaceutical solution comprising acetaminophen. The pharmaceutical solution includes: acetaminophen in an aqueous solution having a pH from about 3.8 up to 6.1; a sufficient amount of a solubilizer; and a weak organic acid having a COOH group and pKa greater than 3. The weak acid is in a concentration from about 1 mg to about 50 mg per 5 ml of the pharmaceutical solution.

### DETAILED DESCRIPTION

In the following description, like reference characters designate like or corresponding parts throughout the several views shown in the figures. It is also understood that terms such as “top,” “bottom,” “outward,” “inward,” and the like are words of convenience and are not to be construed as limiting terms.

Referring to the drawings in general, it will be understood that the illustrations are for the purpose of describing a particular embodiment of the invention and are not intended to limit the invention thereto. As used herein, the following terms have the following meanings:

“A”, “an”, and “the”, as used herein, refers to both singular and plural referents unless the context clearly dictates otherwise.

“About”, as used herein, referring to a measurable value such as a parameter, an amount, a temporal duration, and the like, is meant to encompass variations of +/−20% or less, for example, +/−10%. Thus, for example, the term “about” includes the value given +/−1%, or +/−2%, or +/−3%, or +/−4%, or +/−5%, or +/−6%, or +/−7%, or +/−8%, or +/−9%, or +/−10%, or +/−11%, or +/−12%, or +/−13%, or +/−14%, or +/−15%, or +/−16%, or +/−17%, or +/−18%, or +/−19% or +/−20% of the value specifically disclosed. However, it is to be understood that the value to which the modifier “about” refers is itself also specifically disclosed and included within the value before the term “about” is placed. For example, if the term is “about 3 wt % to about 5 wt %, it is understood that the value can range from 3 wt %+20% to 5 wt %+20%, and that the value includes 3 wt % and 5 wt %.

The term “aqueous” refers to a solution comprising water. In an embodiment, the aqueous solvent is water.

As used herein, the singular shall include the plural and vice versa.

An “effective amount”, as used herein, refers to an amount of acetaminophen sufficient to produce desired biological efficacy or therapeutic effects in a subject being treated, for example, pain relief.

A “therapeutic effective amount” when used in connection with a solution of the present disclosure is an amount sufficient to produce a desired therapeutic result in a subject in need thereof. The actual amount used will vary based on factors such as the type of medical condition, the age, sex, health, species and weight of the patient, and the type of use and length of use, as well as other factors known to those skilled in the art. For example a therapeutic result can include, but is not limited to, treating or preventing pain, nausea or vomiting by a subject.

As used herein, the terms “pharmaceutical solution”, “liquid formulation”, “aqueous composition” and “aqueous formulation” are synonymous and are used interchangeably.

The term acetaminophen, as used herein, includes acetaminophen or N-acetylparaaminophenol and acceptable related variations. The term “solution” as used herein, is a homogeneous mixture as known to one of ordinary skill.

“Stable” means acetaminophen in the aqueous solution retains substantially all of the necessary therapeutic, chemical and/or physical properties associated with acetaminophen for at least about 24 months at room temperature, and in another embodiment, for at least about 36 months at room temperatures or for at least 3 months at the elevated temperature of 40° C. In an embodiment, retaining “substantially all” the necessary properties means to at least 90% of the therapeutic, chemical and/or physical properties of acetaminophen. In another embodiment, stable means the pharmaceutical solution can be stored at least about 24 months at room temperature, and in another embodiment, for at least about 36 months at room temperatures or for at least 3 months at the elevated temperature of 40° C. without the appearance of color and particulate matter which is visible to the human eye.

An embodiment of the invention provides a stable pharmaceutical solution 100 comprising acetaminophen. The pharmaceutical solution includes: an aqueous solution comprising acetaminophen; a sufficient amount of one or more solubilizers; and one or more weak organic acids having a pKa greater than 3. The weak acid is in a sufficient amount to achieve and maintain the pharmaceutical solution at a pH from about 3.8 up to about 6.1 for stability of acetaminophen.

The stable pharmaceutical solution comprising acetaminophen is homogenous in a liquid dosage form, wherein the various components are dissolved and mixed homogenously in liquid dosage form.

Acetaminophen is present in therapeutically effective amounts in embodiments of the pharmaceutical solutions. In one embodiment, the amount of acetaminophen ranges from about 1.0% to about 8 wt % of the pharmaceutical solution, and in another embodiment, from about 1.5 wt % to about 6 wt % of the pharmaceutical solution, and in another embodiment, from about 1.7 wt % to about 5.6 wt % of the pharmaceutical solution. In an embodiment, the concentration of acetaminophen ranges from 100 mg to about 500 mg per 5 ml of the pharmaceutical solution, and in another embodiment from 150 mg to about 450 mg per 5 ml of the pharmaceutical solution, and in another embodiment, from about 200 mg to about 400 mg per 5 ml of the pharmaceutical solution and in another embodiment, about 325 mg per 5 ml of the pharmaceutical solution.

Stabilizing the acetaminophen is the weak organic acid having a pKa value of 3.0 or greater, for example, 3.0, 4.0, 5.0, 6.0. By definition, the pKa value indicates the strength of an acid. pKa is defined as the negative log of the acid dissociation constant or Ka value. It is measured at 25° C. A lower pKa value indicates a stronger acid. That is, the lower value indicates the acid more fully dissociates in water, while a higher pKa value indicates a weaker acid. The weak acid is in a sufficient amount to achieve and maintain the pharmaceutical solution at pH from about 3.8 up to 6.1 for stability of acetaminophen.

Applicant unexpectedly discovered the weak acid can be used as a stabilizing agent. Specifically, Applicant unexpectedly further discovered a minimum amount of weak acid is required to stabilize acetaminophen in solution; but, also not too much contrary to the prior art teachings. A minimum amount of weak acid is needed to buffer or achieve a needed pH; but, too much weak acid as prior art instructs degrades acetaminophen in solution. Applicant surprisingly discovered and solved the perplexity or paradox of adding too little or too much weak acid to stabilize acetaminophen in solution i.e., the weak acid must be higher than a certain amount, but no more than a given amount to stabilize acetaminophen in solution. Applicant surprisingly discovered the lower limit i.e., the minimum effective amount of weak acid needed to achieve a pH (3.81-6.1) to stabilize acetaminophen in solution while also within the upper threshold limit to avoid degradation of acetaminophen in solution.

Not to be bound by theory, the pharmaceutical solution 100 of this invention with the weak acid contained less 4-aminophenol and/or quinoneimine or both compared to aqueous solutions with the same components but without the weak organic acid. For example, an aqueous acetaminophen solution comprising water, acetaminophen and a solubilizing agent had less impurities than the same solution without the weak organic acid present. Even when additional components were added such as, but not limited to, a chelating agent, a sweetening agent, a preservative, etc. the pharmaceutical solution 100 with the weak acid contained less 4-aminophenol and/or quinoneimine or both compared to aqueous solutions with the same components but without the weak organic acid. As a result, the shelf-life of the aqueous solution comprising acetaminophen was enhanced. For example, when the pharmaceutical solution 100 encompassed in this application was stored at room temperature, the pharmaceutical solution 100 was stable for over 45 days, wherein the acetaminophen retained substantially all of its efficacious properties, such as chemical, therapeutic, prophylactic, and physical properties. For example, in an embodiment, the acetaminophen maintained at least 95% of its efficacious properties for at least 36 Months when stored at room temperature.

In an embodiment, the weak organic acid contains 1 to 10 carbon atoms, in another embodiment contains 1-6 carbon atoms, and in another embodiment contains 2-4 carbon atoms. It may be straight-chained or branched, cyclic or aromatic. It may be saturated or unsaturated. It may be a hydroxyl carboxylic acid. As defined herein, the weak organic acids contain only hydrogen atoms, carbon atoms and oxygen atoms. However, the only functional groups present are either hydroxyl groups or carboxy groups. Examples of weak organic acid include, but are not limited to, formic acid, acetic acid, fumaric acid, lactic acid citric acid, malic acid and the like. Thus, as defined, the organic acids exclude such groups as amino acids, thioorganic acids, and lactones.

In an embodiment, the weak organic acid has one or more least one carboxy group or COOH group. In another embodiment, the stable pharmaceutical solution 100 has a plurality of weak acids. The plurality of weak organic acids may be the same or differ from each other. The total amount of weak organic acid may be provided in one unit at one time or increment or repeatedly in multiple units.

If the weak organic acid contains more than one COOH group, it will have more than one pKa value. In such a case, the pKa value that is determinative of whether the acid is within the scope of the disclosure is the first pKa value, that is, the pKa value of the first dissociation constant representing the first dissociation of the hydrogen ion from the organic acid. For example, maleic acid, which is (Z)-butenedioic acid, has two pKa values. The first pKa value for the dissociation of the first hydrogen ion is 1.94 and the pKa for the dissociation of the second hydrogen ion is 6.22. Since the first pKa is less than 3.00, maleic acid is not encompassed within the weak organic acids of the present disclosure. However, the pKa values of its isomer, fumaric acid (E-butenedioic acid) is 3.03 for the first dissociation of hydrogen ion, and the second pKa for the second dissociation of hydrogen ion is 4.44. Since the first pKa value is greater than 3.00, fumaric acid is included within the scope of the weak organic acids contemplated by the present disclosure.

In an embodiment, the weak organic acid contains 1 to 10 carbon atoms, in another embodiment contains 1-6 carbon atoms, and in another embodiment contains 2-4 carbon atoms. It may be straight-chained or branched, cyclic or aromatic. It may be saturated or unsaturated. It may be a hydroxyl carboxylic acid. As defined herein, the weak organic acids contain only hydrogen atoms, carbon atoms and oxygen atoms. However, the only functional groups present are either hydroxyl groups or carboxy groups. Examples of weak organic acid include, but are not limited to, formic acid, acetic acid, fumaric acid, lactic acid citric acid, malic acid and the like. Thus, as defined, the organic acids exclude such groups as amino acids, thioorganic acids, and lactones.

In an embodiment, the weak organic acid has one or more carboxy group or COOH group. In another embodiment, the stable pharmaceutical solution 100 has a plurality of weak acids. The plurality of weak organic acids may be the same or differ from each other. The total amount of weak organic acid may be provided in one unit at one time or increment or repeatedly in multiple units.

If the weak organic acid contains more than one COOH group, it will have more than one pKa value. In such a case, the pKa value that is determinative of whether the acid is within the scope of the disclosure is the first pKa value, that is, the pKa value of the first dissociation constant representing the first dissociation of the hydrogen ion from the organic acid. For example, maleic acid, which is (Z)-butenedioic acid, has two pKa values. The first pKa value for the dissociation of the first hydrogen ion is 1.94 and the pKa for the dissociation of the second hydrogen ion is 6.22. Since the first pKa is less than 3.00, maleic acid is not encompassed within the weak organic acids of the present disclosure. However, the pKa values of its isomer, fumaric acid (E-butenedioic acid) is 3.03 for the first dissociation of hydrogen ion, and the second pKa for the second dissociation of hydrogen ion is 4.44. Since the first pKa value is greater than 3.00, fumaric acid is included within the scope of the weak organic acids contemplated by the present disclosure.

An embodiment of the pharmaceutical solution includes acetaminophen in an aqueous solution having a pH from about 3.8 up to 6.1; a sufficient amount of a solubilizer, and a weak organic acid having a COOH group and pKa greater than 3 and in a range from about 0.01 wt % to about 0.8 wt % of the pharmaceutical solution. A particular embodiment of the pharmaceutical solution includes the weak organic acid in a range from about 0.05 wt % to about 0.5 wt % of the pharmaceutical solution. Another embodiment of the pharmaceutical solution includes acetaminophen in an aqueous solution having a pH from about 3.8 up to 6.1; a sufficient amount of a solubilizer; and a weak organic acid having a COOH group and pKa greater than 3 and in a concentration from about 1 mg to about 50 mg per 5 ml of the pharmaceutical solution. A particular embodiment of the pharmaceutical solution includes citric acid in a concentration from about 1 mg to about 50 mg per 5 ml of the pharmaceutical solution.

In an embodiment, the weak acid is present in sufficient amount to achieve and maintain the pharmaceutical solution at a pH of from about 3.8 up to 6.1 but low enough to minimize degradation of acetaminophen. In an embodiment, the amount of weak organic acid present in the pharmaceutical solution ranges from about 0.01 to about 0.8 wt %, and in another embodiment, from about 0.05 to about 0.5 wt % and in further embodiment, from about 0.1 to about 0.2 wt %. In an embodiment, the concentration of weak acid ranges from about 1 mg to about 50 mg per 5 ml of the pharmaceutical solution; in another embodiment, from about 3 mg to about 30 mg per 5 ml of the pharmaceutical solution; and in further embodiment, and from about 0.5 mg to about 15 mg per 5 ml of the pharmaceutical solution. In an embodiment, the weight ratio of the weak organic acid to acetaminophen ranges from about 1:325 to about 1:7, and in another embodiment, from about 1:100 to about 1:10, and in a further embodiment, from about 1:50 to about 1:20. In a particular embodiment, the weak acid includes citric acid; and the concentration of citric acid ranges from about 1 mg to about 50 mg per 5 ml of the pharmaceutical solution; in another embodiment, from about 3 mg to about 30 mg per 5 ml of the pharmaceutical solution; in another embodiment, from about 0.5 mg to about 15 mg per 5 ml of the pharmaceutical solution; and in another embodiment, about 8 mg to about 9 mg per 5 ml of the pharmaceutical solution.

The pH of the pharmaceutical solution ranges from about 3.0 to about 6.5, and in another embodiment, from about 4.0 to about 6.5, and in a further embodiment, from about 4.4 to about 5.9. In an embodiment, the pH is about 5.60. Salt such as Sodium Citrate may also be used to further adjust and maintain the pH. To maintain the pH, buffers may be present in the pharmaceutical solution. The buffer can help maintain pH of the pharmaceutical solution. In an example, the pharmaceutical solution comprises from about 0.05 wt % to about 2 wt % buffer; in another example from about 0.1 wt % to about 1 wt % buffer; and in another example from about 0.15 wt % to about 0.5 wt % buffer. Buffers can include acetate buffers, citrate buffers, and phosphate buffers. Non-limiting examples of buffers can include acetic acid/sodium acetate; citric acid/sodium citrate; monobasic sodium phosphate/dibasic sodium phosphate; sodium carbonate/sodium bicarbonate; succinic acid/sodium succinate; potassium dihydrogen phosphate/phosphoric acid, etc. either individually or in combinations of two or more thereof.

In an embodiment, one or more buffers is present. In a particular embodiment, buffer system includes one or more salts such as Sodium Citrate; and one or more weak organic acids may further optionally be added to the buffer system with salts. For example: in a buffer system with one or more salts, citric acid may optionally be added. If the weak organic acid is added to the buffer, the amount of organic acid added is low enough to not degrade acetaminophen in the solution but sufficiently high enough to provide needed pH and buffer capacity.

The pharmaceutical solution 100 also comprises one or more solubilizing agents present in amounts effective to solubilize the acetaminophen in an aqueous medium. As used herein, the solubilizing agent is a water soluble polymer. Examples include povidone (Kollidon 12PF or Kollidon 17PF), poloxamer, polyethylene glycol, such as polyethylene 400 and polysorbate, such as polysorbate 20, polysorbate 40, polysorbate 80, macrogol 200, and macrogol 400 and a mixture thereof. For example, in one embodiment, the polyethylene glycol is the solubilizing agent. The polyethylene glycol in this embodiment is the low molecular weight polyethylene glycol. If present, the molecular weight of the polyethylene glycol ranges from about 200 g/mole to about 800 g/mole, and in another embodiment from about 300 g/mole to about 600 g/mole, and in a further embodiment, about 400 g/mole. In an embodiment, polysorbate is the solubilizing agent. In this embodiment, a low molecular weight polysorbate is used. For example, if polysorbate is the solubilizing agent, the molecular weight of the polysorbate used ranges from about 1000 g/mole to about 1500 g/mole and in another embodiment form about 1100 g/mole to about 1400 g/mole. Without wishing to be bound, it is believed that the solubilizing agent forms a hydrogen bond with acetaminophen in the pharmaceutical solution to increase solubility, and blocks the intermolecular bond of acetaminophen with other acetaminophen molecules to prevent a generation of re-crystallization or precipitation. In addition, the solubilizing agent functions to delay generation of recrystallization at a temperature of room temperature or less.

The solubilizing agent is present in the amount of about 10 wt % to about 70 wt %, and in another embodiment, from about 15 wt % to about 40 wt %, and in another embodiment, from about 20 wt % to about 30 wt % of the pharmaceutical solution. In an embodiment, the solubilizing agent is present in the amount of about 10 wt % to about 70 wt %, and in another embodiment, from about 20 wt % to 45 wt %, and in another embodiment, from about 25 wt % to about 35 wt % per 5 mL of the pharmaceutical solution. When the content of the solubilizing agent is too low, acetaminophen may not be sufficiently dissolved in the aqueous pharmaceutical solution, and in particular, solubility of acetaminophen is lowered at a low temperature, and thus a precipitate may be formed. When the content of the solubilizing agent is too large, the solubilizing agent itself may not be sufficiently dissolved in the pharmaceutical solution. In an embodiment, the weight ratio of solubilizing agent to acetaminophen ranges from about 1.7 to about 11, and in another embodiment, from about 3.5 to about 6.5, and in a further embodiment, from about 4 to about 6.

It should be appreciated that the pharmaceutical solution also includes the reaction product of the acetaminophen, weak acid, and solubilizer with each other, individually or in combinations with each other.

The pharmaceutical solution 100 may optionally include one or more components known and suitable in the pharmaceutical arts, either individually or in combinations of two or more thereof. Non-limiting examples of components, for illustration and not limitation, include such sweeteners, flavorings agents, coloring agents, thickeners, chelating agents, anti-oxidants, preservatives known and suitable in the pharmaceutical arts, either individually or in combinations of two or more thereof. For example: Acetaminophen displays bitter, acrid and sour notes; thus, sweeteners and/or flavoring agents or both may be added to mask the bitter, acrid and sour notes of acetaminophen.

In an embodiment, the pharmaceutical solution 100 further includes one or more preservatives. In a non-limiting example, the pharmaceutical solution optionally comprises from about 0.01 wt % to about 1% preservative, in another example from about 0.05% to about 0.8% preservative, in another example from about 0.07% to about 0.7% preservative, and in another example from about 0.08% to about 0.25% preservative. Non-limiting examples of preservatives include benzalkonium chloride, ethylenediaminetetraacetic acid (EDTA), benzyl alcohol, potassium sorbate, parabens, benzoic acid, sodium benzoate, and mixtures thereof. In an embodiment, the stable pharmaceutical solution 100 may have a plurality of one or more respective components described supra, such as a plurality solublizers, sweeteners, flavorings agents, coloring agents, thickeners, chelating agents, anti-oxidants, preservatives, etc. When a pharmaceutical solution 100 has a plurality of a respective component, the component may be the same or differ from each other. The total sum amount of a component may be provided in one unit at one time or increment or repeatedly in multiple units.

Optionally, the pharmaceutical solution 100 may include one or more medicaments/pharmaceuticals. Non-limiting examples of medicament include such as CNS-acting analgesics, MSR (multi-symptom relief) cold/flu active medicaments, either individually or in combinations of two or more thereof. The one or more optional medicaments may be in therapeutic effective concentrations; and not limited by such.

For example: the pharmaceutical solution 100 may include one or more CNS-acting analgesic such as morphinic analgesic. Examples of morphines include such as, but not limited to, morphinic derivatives of natural, semi-synthetic or synthetic origin and piperidine derivatives from the following either individually or in combinations of two of more thereof: buprenorphine, dramadol, codeine, oxycodone, dextromoramide, dextropropoxyphene, hydrocodone, hydromorphone, ketobemidone, levomethadone, levorphanol, meptazinol, methadone, morphine, nalbuphine, nicomorphine, dizocine, diamorphine, dihydrocodeine, dipipanone, methorphane, dextromethorphane and, where applicable, pharmaceutically acceptable salts thereof. In an embodiment, the codeine or codeine derivative concentration, expressed in terms of codeine base, comprises from about 0.04% and 25 wt %; and in another embodiment, from about 0.05% to about 15 wt % in relation to the acetaminophen content. In an embodiment, morphine or morphine derivative concentration, expressed in terms of morphine base comprises from about 0.05 to about 15 wt %; and in another embodiment from about 0.08 to about 5 wt % in relation to acetaminophen content. In an embodiment, the morphine or morphine derivative concentration, expressed in terms of morphine base, comprises from about 0.1 mg/ml to about 20 mg/ml; in another embodiment, from about 0.5 mg/ml to about 10 mg/ml; in another embodiment, from about 0.75 mg to about 5 mg/ml; and in another further embodiment, at about 1 mg/ml of the oral liquid formulation.

In an embodiment, the oral pharmaceutical solution also comprises oxycodone salts, such as oxycodone HCl, in the amounts described hereinabove. In an embodiment, the oxycodone HCl concentration comprises from about 0.04% and 25 wt %; in another embodiment from about 0.05% to about 15 wt %; and in anther embodiment from about 0.08 to about 5 wt % in relation to acetaminophen content. In an embodiment, the oxycodone HCl concentration comprises from about 0.1 mg/ml to about 20 mg/ml; in another embodiment, from about 0.5 mg/ml to about 10 mg/ml; and in another embodiment, from about 0.75 mg to about 5 mg/ml; and in another embodiment, at about 1 mg/ml of the pharmaceutical solution.

In an embodiment, the pharmaceutical solution 100 has 0.9 wt % oxycodone HCl in combination with acetaminophen. i.e., total amount of oxycodone HCl and acetaminophen is 0.9 wt % of the pharmaceutical solution 100. In another embodiment, the pharmaceutical solution has an oxycodone HCL concentration of about 5 mg/5 ml of the pharmaceutical solution.

Regardless of the number of components that comprise the pharmaceutical solution of the present disclosure, the sum of the weights of all of the components of the present disclosure add up to 100% by weight.

In an embodiment, the pharmaceutical solution also includes “aqueous-based orally acceptable pharmaceutical carrier” consistent with conventional pharmaceutical practices and well known to those skilled in the art. The amount of water in the pharmaceutical solution 100 can vary over quite a wide range depending upon the total weight and volume of the active component and other optional non-active components as known to one of ordinary skill. In an embodiment, the total water content, based on the weight of the final pharmaceutical solution, will generally range from about 30 to about 70%, and, in another embodiment from about 35 to about 65%, by weight/weight. In an embodiment, the water content of the pharmaceutical solution is about 50 w/v % of the total solution or higher, and in another embodiment, about 45 w/v % or higher, and in a further embodiment, about 60 w/v % or higher relative to the total volume of the pharmaceutical solution. Methods for preparations and manufacture of solutions, suspensions, and emulsions are discussed in Remington's Pharmaceutical Sciences (Alfonso R. Gennaro, editor), pp. 1519, incorporated herein by reference.

Although water itself may make up the entire carrier, typical liquid formulations preferably contain a co-solvent, for example, propylene glycol, corn syrup, glycerin, sorbitol solution and the like, to assist solubilization and incorporation of water-insoluble components, such as flavoring oils and the like into the pharmaceutical solution. In general, therefore, the pharmaceutical solutions of this disclosure may contain from about 1 to about 70% v/v and, most preferably, from about 5 to about 50% v/v, of the co-solvent.

The invention also encompasses methods of making the stable pharmaceutical solution comprising 100 acetaminophen and not limited by the order unless stated otherwise. In one embodiment, the method includes providing water, acetaminophen, the solubilizing agent, weak organic acid as described supra in sufficient amount to form and maintain the solution at a pH from about 3.8 up to 6.61 for stability of acetaminophen. The method is not limited by the order or sequence of when and how water, acetaminophen, the solubilizing agent, and weak organic acid such as citric acid are provided. Any other component as described above may also be provided.

In a particular embodiment, the method includes mixing the water, acetaminophen, a solubilizing effective amount of a solubilizer, and weak organic acid like citric acid at or about a temperature ranging from about 20° C. to about 30° C.; and while in another embodiment, the mixing takes place at about room temperature. The method is not limited by a sequential order of providing water, acetaminophen, citric acid, the solubilizing agent and any other component provided. Furthermore, the water, acetaminophen, citric acid, the solubilizing agent may be sequentially or simultaneously provided. The method is also not limited by frequency. In an embodiment, the method further including providing salt such as Sodium Citrate to further adjust and maintain the pH as needed. In an embodiment, method includes providing one or more buffers such as salts and as described above to maintain the pH of the pharmaceutical solution.

In a particular embodiment, the method including providing buffer such as Sodium Citrate; and optionally also adding citric acid. If the weak organic acid is provided, the amount of organic acid used is low enough to not degrade acetaminophen in the solution but sufficiently high enough to provide needed pH and buffer capacity.

The invention also encompasses methods of treating a disease or condition that is responsive to acetaminophen (e.g., pain and/or fever). In an embodiment, the method of treating a disease or condition includes administering to a subject in need thereof an effective amount of the acetaminophen pharmaceutical solution described above. The invention encompasses administering the acetaminophen pharmaceutical solution 100 for various disease or condition such as pain, fever, inflammation, ischemic injury (such as myocardial and/or cerebral), or neuronal injury, etc.; and not limited by the disease or condition. Invention encompasses administering an effective amount of the aqueous liquid acetaminophen pharmaceutical solution for one or more diseases or conditions and/or symptoms thereof as determined by a skilled physician routine in the art and not limited by dosing regimen (e.g., detection of allergies, cold, cough, flu, pain, etc.). The method may also further include determining and monitoring the response or responses from administering the pharmaceutical solution.

In some embodiments, the subject includes a mammal; and the term “subject” is not restricted to a specific animal or mammal and does not denote a particular age or sex. In some embodiments, the mammal is a primate. In some embodiments, the primate is a human. In some embodiments, the subject is human, including adults, children, infants, and preemies. In some variations, the primate is a non-human primate such as chimpanzees and other apes and monkey species. In some embodiments, the mammal is a farm animal such as cattle, horses, sheep, goats, and swine; pets such as rabbits, dogs, and cats; laboratory animals including rodents, such as rats, mice, and guinea pigs; and the like.

The invention is not limited by how administered and how much. Embodiments of the invention encompasses administering various dosing regimens of the acetaminophen pharmaceutical solution as deemed effective and within the capabilities of those skilled in the art. The acetaminophen pharmaceutical solution may be formulated and/or administered in conjunction with one or more additional pharmaceutical agents, as known to one of skill in the art. The acetaminophen pharmaceutical solution and one or more agents may be administered simultaneously or sequentially (e.g., prior to, concurrently with, or after).

Embodiments of the invention also encompass kits having materials useful for the treatment or prevention of a condition that is responsive to acetaminophen (e.g., pain). The kits include a stable pharmaceutical solution 100 of acetaminophen as described supra and instructions for use. The kits may include a container with a label with directions for use, such as those described herein. The kit may also include a dropper and/or another measuring unit for appropriate dosage; and package inserts with directions for administration, dosages, as well as safety precautions

The following non-limiting examples further illustrate the invention described herein.

### EXAMPLES

### Examples 1-4

The Pharmaceutical formulation utilized in the following examples contains an aqueous solution of Acetaminophen, a solubilizing agent containing polyethylene glycol, a chelating agent containing EDTA, sweetening system containing Sucralose, a preservative containing sodium benzoate and a weak organic acid containing citric acid. The liquid formulations of the examples were conducted at room temperature and contain an amount of Acetaminophen between 100 mg to 500 mg per 5 ml of the formulation.

Table 1 provides the contents of the pharmaceutical solutions that were used in each of the examples:

TABLE 1

| | | Example 1 | Example 2 | Example 3 | Example 4 |
|---|---|---|---|---|---|
| Sr | Ingredient | mg/5 ml | mg/5 ml | mg/5 ml | mg/5 ml |
| 1 | Acetaminophen | 325.00 | 325.00 | 325.00 | 325.00 |
| 2 | Polyethylene | 1734.00 | 1734.00 | 1734.00 | 1734.00 |
| | Glycol 400 | | | | |
| 3 | Disodium EDTA | 2.31 | 2.31 | 2.31 | 2.31 |
| 4 | Fructose | 1156.00 | 1156.00 | 1156.00 | 1156.00 |
| 5 | Sodium Benzoate | 11.56 | 11.56 | 11.56 | 11.56 |
| 6 | Sucralose | 13.87 | 13.87 | 13.87 | 13.87 |
| 7 | Citric acid | 37.57 | 2.90 | — | 14.20 |
| | Anhydrous | | | | |
| 8 | Color | 1.16 | 1.16 | 1.16 | 1.16 |
| 9 | Purified water | q.s. | q.s. | q.s. | q.s. |
| | up to 100% w/w | | | | |

The pharmaceutical solutions of Examples 1-4 were prepared by adding to water, polyethylene glycol 400, Acetaminophen, anhydrous citric acid, Sucralose, sodium EDTA, sodium benzoate, fructose and color and mixed to form a homogenous product at room temperature.

Table 2 provides the initial data of pH and Impurity profile. Example 3 without citric acid has a pH of 6.59 outside the USP Monograph pH limit −3.8 to 6.1. The other examples 1, 2, and 4 with citric acid have pH within acceptable guidelines. Imide A is as follows:

### Chemical Name—[3-Hydroxy-1-(4-hydroxy-phenyl)-2,5-dioxo-pyrrolidin-3-yl]-acetic acid Molecular Formula—C₁₂H₁₁NO₆

TABLE 2

| Initial Data | | Example 1 | Example 2 | Example 3 | Example 4 |
|---|---|---|---|---|---|
| pH | | 3.78 | 5.59 | 6.59 | 4.54 |
| Impurity | Imide A | ND | ND | ND | ND |
| Profile | 4-Amino | 0.014% | 0.005% | ND | ND |
| | phenol | | | | |
| | Unknown | ND | ND | ND | ND |
| | impurity | | | | |
| | Total | 0.014% | 0.005% | 0.000% | 0.000% |
| | impurity | | | | |

Note:
ND: Not Detected

The prepared samples were exposed to 40° C./75% RH condition for 90 days. Sample with higher concentration of citric acid (37.57 mg/5 mL) showed higher levels of 4-amino phenol and Imide A impurity.

Table 3 contains the data of Impurity profile of experiments at the end of 90 days.

TABLE 3

| Exposed 90 days 40° C./75% RH | | Example 1 | Example 2 | Example 3 |
|---|---|---|---|---|
| Impurity | Imide A | 0.010% | ND | ND |
| Profile | 4-Amino | 0.370% | 0.010% | 0.010% |
| | phenol | | | |
| | Unknown | 0.010% | ND | 0.001% |
| | impurity 1 | | | |
| | Unknown | 0.040% | ND | ND |
| | impurity 2 | | | |
| | Unknown | 0.420% | 0.010% | 0.003% |
| | impurity 3 | | | |
| | Unknown | 0.010% | ND | ND |
| | impurity 4 | | | |
| | Unknown | 0.020% | ND | 0.003% |
| | impurity 5 | | | |
| | Unknown | 0.010% | ND | ND |
| | impurity 6 | | | |
| | Unknown | ND | ND | 0.001% |
| | impurity 7 | | | |
| | Unknown | ND | ND | 0.002% |
| | impurity 8 | | | |
| | Total | 0.401% | 0.020% | 0.020% |
| | impurity | | | |

Note:
ND: Not Detected

Samples were also exposed to stress conditions of 80° C. for 24 hours and the results are shown in Table 4. Samples with higher concentrations of citric acid showed higher level of 4-amino phenol and Imide A impurity.

Table 4 contains data of stress study.

TABLE 4

| Exposed 24 hours 80° C. | | Example 1 | Example 2 | Example 3 | Example 4 |
|---|---|---|---|---|---|
| Impurity | Imide A | 0.014% | ND | ND | 0.004% |
| Profile | 4-Amino | 0.159% | ND | ND | 0.022% |
| | phenol | | | | |
| | Unknown | 0.003% | ND | ND | ND |
| | impurity 1 | | | | |
| | Unknown | 0.023% | ND | ND | 0.004% |
| | impurity 2 | | | | |
| | Unknown | 0.161% | ND | ND | 0.012% |
| | impurity 3 | | | | |
| | Unknown | ND | ND | ND | ND |
| | impurity 4 | | | | |
| | Unknown | ND | ND | ND | ND |
| | impurity 5 | | | | |
| | Unknown | ND | ND | ND | ND |
| | impurity 6 | | | | |
| | Total | 0.361% | 0.000% | 0.000% | 0.042% |
| | impurity | | | | |

Note:
ND: Not Detected

Table 5 contains data of antimicrobial effectiveness test.

TABLE 5

| 28^{th} day count | Example 1 | Example 2 |
|---|---|---|
| Escherichia Coli | Nil | Nil |
| Staphylococcus aureus | Nil | Nil |
| Pseudomonas aeruginosa | Nil | Nil |
| Candida albicans | Nil | Nil |
| Aspergillus brasiliensis | Nil | Nil |

From Examples 1, 2, 3 and 4, it is seen that as the concentration of citric acid increased in the formulation, the pH of the solution decreased, and Imide A impurity level increased. But as pH increased from 3.78 to 5.59, the stability of the product improved.

To balance the stability and preservative effectiveness, the product was adjusted to a pH of about 5.59 with a weak organic acid such as citric acid.

The above preferred embodiments and examples were given to illustrate the scope and spirit of the present invention. These embodiments and examples will make apparent to those skilled in the art other embodiments and examples. The other embodiments and examples are within the contemplation of the present invention. Therefore, the present invention should be limited only by the amended claims.

What is claimed is:

1. A pharmaceutical solution comprising:
acetaminophen in an aqueous solution having a pH from about 3.8 up to 6.1;
a sufficient amount of a solubilizer; and
a weak organic acid having a having a COOH group and pKa greater than 3 in a sufficient amount to achieve and maintain the pH of solution from about 3.8 up to 6.1 for stability of acetaminophen.

2. The pharmaceutical solution of claim 1 wherein the weak organic acid comprises at least one of citric acid, fumaric acid, acetic acid, lactic acid, malic acid and combinations thereof.

3. The pharmaceutical solution of claim 2, wherein the concentration of weak organic acid comprises citric acid.

4. The pharmaceutical solution of claim 1, wherein the weak organic acid ranges from about 0.01 wt % to about 0.8 wt % of the pharmaceutical solution.

5. The pharmaceutical solution of claim 1, wherein the weak organic acid ranges from about 0.05 wt % to about 0.5 wt % of the pharmaceutical solution.

6. The pharmaceutical solution of claim 1, wherein the weak organic acid ranges from about 0.1 wt % to about 0.2 wt % of the pharmaceutical solution.

7. The pharmaceutical solution of claim 1, wherein the concentration of acetaminophen ranges from about 100 mg/5 ml to about 500 mg/5 ml of the pharmaceutical solution.

8. The pharmaceutical solution of claim 1, wherein the concentration of acetaminophen ranges from about 150 mg/5 ml to about 450 mg/5 ml of the pharmaceutical solution.

9. The pharmaceutical solution of claim 1, wherein the concentration of acetaminophen ranges from about 200 mg/5 ml to about 400 mg/5 ml of the pharmaceutical solution.

10. The pharmaceutical solution of claim 1 wherein the pH is from about 4 up to 6.1.

11. The pharmaceutical solution of claim 1, further comprises another second pharmaceutical.

12. The pharmaceutical solution of claim 11, wherein the second pharmaceutical comprises an analgesic.

13. The pharmaceutical solution of claim 1, further comprises a second pharmaceutical comprising oxycodone.

14. The pharmaceutical solution of claim 11, wherein the additional pharmaceutical comprises at least one a decongestant, expectorant, antihistamine, antitussive, pain reliever, or combinations thereof.

15. A method of making a pharmaceutical solution comprising:
mixing water, acetaminophen, a sufficient amount of a solubilizer, and a weak organic acid having a COOH group and pKa greater than 3 in a sufficient amount to form and maintain the solution at a pH from about 3.8 up to 6.1 for stability of acetaminophen.

16. The method of claim 15, wherein the weak organic acid comprises at least one of citric acid, fumaric acid, acetic acid, lactic acid, malic acid, and combinations thereof.

17. The method of claim 15, wherein the weak organic acid comprises citric acid.

18. The method of claim 15, wherein the weak organic acid ranges from about 0.01 wt % to about 0.8 wt % of the pharmaceutical solution.

19. The method of claim 15, further comprises a second pharmaceutical comprising oxycodone.

20. A pharmaceutical solution comprising:
an aqueous solution comprising acetaminophen;
a solubilizing effective amount of a solubilizer;
a weak organic acid comprising citric acid having a pKa greater than 3, in a sufficient amount to achieve and maintain the solution at a pH from about 4.5 up to 6.1 for stability of acetaminophen; and
a second pharmaceutical comprising oxycodone from about to about 0.08 to about 5 wt % in relation to the acetaminophen content.
